SEQUENCE LISTING (31 sequences)

SEQ ID NO: 1 - DNA, 17 bp, synthetic construct
  source 1..17: mol_type = other DNA
cgcgctcggcttccact

SEQ ID NO: 2 - DNA, 19 bp, synthetic construct
  source 1..19: mol_type = other DNA
agaccacgcgcggcatcag

SEQ ID NO: 3 - DNA, 18 bp, synthetic construct
  source 1..18: mol_type = other DNA
gcgcgagtcgcccatgtc

SEQ ID NO: 4 - DNA, 19 bp, synthetic construct
  source 1..19: mol_type = other DNA
agcgtggcggtaaagttct

SEQ ID NO: 5 - DNA, 19 bp, synthetic construct
  source 1..19: mol_type = other DNA
cgtagtacagcaggcaccg

SEQ ID NO: 6 - DNA, 16 bp, synthetic construct
  source 1..16: mol_type = other DNA
acgagccccgcttcca

SEQ ID NO: 7 - DNA, 18 bp, synthetic construct
  source 1..18: mol_type = other DNA
agatgcagggctcgtaca

SEQ ID NO: 8 - DNA, 18 bp, synthetic construct
  source 1..18: mol_type = other DNA
tgtccccgggcgagaaga

SEQ ID NO: 9 - DNA, 31 bp, synthetic construct
  source 1..31: mol_type = other DNA
atcaggtcgaacgtgtccccgggcgagaaga

SEQ ID NO: 10 - DNA, 20 bp, synthetic construct
  source 1..20: mol_type = other DNA
ggggacacgttcgacctgat

SEQ ID NO: 11 - DNA, 35 bp, synthetic construct
  source 1..35: mol_type = other DNA
agaccacgcgcggcatcaggcgctcggcttccact

SEQ ID NO: 12 - DNA, 41 bp, synthetic construct
  source 1..41: mol_type = other DNA
gagaactttaccgccacgctggcgtagtacagcaggcaccg

SEQ ID NO: 13 - DNA, 24 bp, synthetic construct
  source 1..24: mol_type = other DNA
ttatcagatacctatgcataccca

SEQ ID NO: 14 - DNA, 24 bp, synthetic construct
  source 1..24: mol_type = other DNA
tgaacatgagcttttctttatcgc

SEQ ID NO: 15 - DNA, 18 bp, synthetic construct
  source 1..18: mol_type = other DNA
aacatcatcttcccgata

SEQ ID NO: 16 - DNA, 22 bp, synthetic construct
  source 1..22: mol_type = other DNA
tccgggtaatttcttcaacatc

SEQ ID NO: 17 - DNA, 20 bp, synthetic construct
  source 1..20: mol_type = other DNA
ctggatgatgattggttcag

SEQ ID NO: 18 - DNA, 18 bp, synthetic construct
  source 1..18: mol_type = other DNA
gaagggacgctatgtcga

SEQ ID NO: 19 - DNA, 24 bp, synthetic construct
  source 1..24: mol_type = other DNA
tacaaataatcgcccgtagctgat

SEQ ID NO: 20 - DNA, 48 bp, synthetic construct
  source 1..48: mol_type = other DNA
acatgagcttttctttatcgcattttatcagatacctatgcataccca

SEQ ID NO: 21 - DNA, 48 bp, synthetic construct
  source 1..48: mol_type = other DNA
cagaccatgtctgtatatgctgcctgtaagatcaatatcattttccgg

SEQ ID NO: 22 - DNA, 20 bp, synthetic construct
  source 1..20: mol_type = other DNA
tggtgaacgtgtccgagggc

SEQ ID NO: 23 - DNA, 20 bp, synthetic construct
  source 1..20: mol_type = other DNA
cgggcaggaacgtccagatc

SEQ ID NO: 24 - DNA, 17 bp, synthetic construct
  source 1..17: mol_type = other DNA
ggccctcgcatccctga

SEQ ID NO: 25 - DNA, 16 bp, synthetic construct
  source 1..16: mol_type = other DNA
acgcggtctcgaagca

SEQ ID NO: 26 - DNA, 18 bp, synthetic construct
  source 1..18: mol_type = other DNA
tccactcgcagctcttct

SEQ ID NO: 27 - DNA, 18 bp, synthetic construct
  source 1..18: mol_type = other DNA
cgctggctgttatcagat

SEQ ID NO: 28 - DNA, 20 bp, synthetic construct
  source 1..20: mol_type = other DNA
ctatgcatacccaacatcag

SEQ ID NO: 29 - DNA, 200 bp, synthetic construct
  source 1..200: mol_type = other DNA
gccgcgcgggcttcggctcggccctcgcatccctgagggaggcgcccccggcccatctggtgaacgtgtccgagggcgccaacttcaccctcgacgcgcgcggcgacggcgccgtgctggccgggatctggacgttcctgcccgtccgcggctgcgacgccgtgtcggtgaccacggtgtgcttcgagaccgcgtgccac

SEQ ID NO: 30 - DNA, 1740 bp, synthetic construct
  source 1..1740: mol_type = other DNA
atgcggccctttctgctgcgcgccgcgcagctcctggcgctgctggccctggcgctctccaccgaggccccgagcctctccgccgagacgaccccgggccccgtcaccgaggtcccgagtccctcggccgaggtctgggacgacctctccaccgaggccgacgacgatgacctcaacggcgacctcgacggcgacgaccgccgcgcgggcttcggctcggccctcgcatccctgagggaggcgcccccggcccatctggtgaacgtgtccgagggcgccaacttcaccctcgacgcgcgcggcgacggcgccgtgctggccgggatctggacgttcctgcccgtccgcggctgcgacgccgtgtcggtgaccacggtgtgcttcgagaccgcgtgccacccggacctggtgctgggccgcgcctgcgtccccgaggccccggagatgggcatcggcgactacctgccgcccgaggtgccgcggctccggcgcgagccgcccatcgtcaccccggagcggtggtcgccgcacctgagcgtcctgcgggccacgcccaacgacacgggcctctacacgctgcacgacgcctcggggccgcgggccgtgttctttgtggcggtgggcgaccggccgcccgcgccggcggacccggtgggccccgcgcgccacgagccccgcttccacgcgctcggcttccactcgcagctcttctcgcccggggacacgttcgacctgatgccgcgcgtggtctcggacatgggcgactcgcgcgagaactttaccgccacgctggactggtactacgcgcgcgcgcccccgcggtgcctgctgtactacgtgtacgagccctgcatctaccacccgcgcgcgcccgagtgcctgcgcccggtggacccggcgtgcagcttcacctcgccggcgcgcgcgcggctggtggcgcgccgcgcgtacgcctcgtgcagcccgctgctcggggaccggtggctgaccgcctgccccttcgacgccttcggcgaggaggtgcacacgaacgccaccgcggacgagtcggggctgtacgtgctcgtgatgacccacaacggccacgtcgccacctgggactacacgctcgtcgccaccgcggccgagtacgtcacggtcatcaaggagctgacggccccggcccgggccccgggcaccccgtggggccccggcggcggcgacgacgcgatctacgtggacggcgtcacgacgccggcgccgcccgcgcgcccgtggaacccgtacggccggacgacgcccgggcggctgtttgtgctggcgctgggctccttcgtgatgacgtgcgtcgtcgggggggccatctggctctgcgtgctgtgctcccggcgccgggcggcctcgcggccgttccgggtgccgacgcgggcgcggacgcacatgctctctccggtgtacaccagcctgcccacgcacgaggactactacgacggcgacgacgacgacgacgaggaggcgggcgtcatccgccggcggcccgcctcccccggcggagacagcggctacgaggggccgtacgcgagcctggaccccgaggacgagttcagcagcgacgaggacgacgggctgtacgtgcgccccgaggaggcgccccgctccggcttcgacgtctggttccgcgatccggagaaaccggaagtgacgaatggacccaactatggcgtgaccgccaaccgcctgttgatgtcccgccccgcttaa

SEQ ID NO: 31 - DNA, 250 bp, synthetic construct
  source 1..250: mol_type = other DNA
atcgctttcctggccctggatgatgattggttcagcgctggctgttatcagatacctatgcatacccaacatcagctacgggcgattatttgtaataaatgcgataaagaaaagctcatgttcagaccatgtctgtatatgctgccgcatatttatcgggaagatgatgttgaagaaattacccggaaaatgatattgatcttacataaacgagcgcttcgacatagcgtcccttctggcatttgccact

(19) United States
(12) Patent Application Publication
(10) Pub. No.: US 2024/0368679 A1
(43) Pub. Date: Nov. 7, 2024

(54) LINEAR DISPLACEMENT ISOTHERMAL AMPLIFICATION METHOD AND APPLICATION THEREOF

(71) Applicant: South China Agricultural University, Guangzhou (CN)

(72) Inventors: Ming LIAO, Guangzhou (CN); Jianmin ZHANG, Guangzhou (CN); Qijie LIN, Guangzhou (CN); Hongchao GOU, Guangzhou (CN); Kaiyuan JIA, Guangzhou (CN); Junhao PENG, Guangzhou (CN); Yucen LIANG, Guangzhou (CN)

(21) Appl. No.: 18/775,402

(22) Filed: Jul. 17, 2024

Related U.S. Application Data
(63) Continuation of application No. PCT/CN2023/119324, filed on Sep. 18, 2023.

(30) Foreign Application Priority Data
Sep. 30, 2022 (CN) 202211206642.8

(51) Int. Cl. C12Q 1/6844

(52) U.S. Cl. CPC C12Q 1/6844; C12Q 2600/16

(57) ABSTRACT

A linear displacement isothermal amplification (LDIA) method and application thereof are by the present disclosure. The LDIA method of the present disclosure specifically starts the initial reaction of LDIA for four common primers of the template, including a pair of external primers (LOF and LOR) and internal primers (LIF and LIR), and an accelerating primer (LAR) may also be added in the reaction to form a short sequence product. The method provided by the disclosure greatly reduce the difficulty of primer design while maintaining the sensitivity and specificity similar to other isothermal amplification reactions such as loop-mediated isothermal amplification methods.

### CROSS-REFERENCE TO RELATED APPLICATIONS

This application is a continuation of PCT/CN2023/119324, filed Sep. 18, 2023 and claims priority of Chinese Patent Application No. 202211206642.8, filed on Sep. 30, 2022, the entire contents of which are incorporated herein by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: US-2024-2937-Sequence
Creation date: Jul. 3, 2024
Byte size: 30,430

### TECHNICAL FIELD

The present disclosure belongs to the technical field of isothermal amplification, and in particular to a linear displacement isothermal amplification method and application thereof.

### BACKGROUND

In the field of life sciences, nucleic acid amplification is a widely used technique for clinical disease diagnosis, and polymerase chain reaction (PCR) is one of the most common nucleic acid amplification methods with relatively simple primer design, allowing easy application in the detection of various pathogens. However, PCR is difficult to apply to field detection due to the requirements of reaction temperature and reaction instrumentation. Compared to PCR, isothermal amplification methods, such as loop-mediated isothermal amplification (LAMP), cross-primer amplification (CPA), recombinase polymerase amplification (RPA), etc., have a great advantage in field testing because they do not require complex temperature control instruments. Nevertheless, the amplification range is limited by strict target requirements and complicated primer design. Taking LAMP as an example, the amplification efficiency of LAMP is severely compromised when faced with fragments less than 200 bp in length, or fragments with too high or too low a GC content. In addition, multiple primer pairs and complex stem-loop structures may lead to the formation of primer dimers, resulting in non-specific amplification and false positives.

Therefore, it is of great practical value and research importance to establish an isothermal amplification method capable of detecting nucleic acid fragments with different lengths and GC contents, and to simplify the primer design.

### SUMMARY

The present disclosure provides an isothermal amplification method capable of detecting nucleic acid fragments of different lengths and GC contents and has simpler primer design.

The technical scheme adopted by the present disclosure is as follows.

One aspect of the present disclosure provides a linear displacement isothermal amplification (LDIA) method, specifically including following steps:
S1, hybridizing an external primer LOF, an external primer LOR, an internal primer LIF and an internal primer LIR with a target sequence to form single-stranded DNA under catalysis of an external primer and polymerase, and forming short double-stranded DNA under an action of an internal primer;
S2, allowing for dynamic dissociation of short double-stranded DNA and amplification to form new amplification products catalyzed by internal primers and polymerases; and
S3, repeating the S1 and the S2 repeatedly to obtain a large number of amplification products.

Optionally, an accelerating primer LAR is added in the S1; the LAR is positioned between LIF and LIR and a region of LAR binding to a target does not overlap with a region of LIF and LIR binding to the target, where the accelerating primer LAR is further amplified with the internal primer LIF or the internal primer LIR to form an amplification product with a shorter sequence.

Optionally, a molar ratio of the external primer LOF, the external primer LOR, the internal primer LIF, the internal primer LIR and the accelerating primer LAR is (1-2):(1-2):(4-10):(4-10):(3-4).

Optionally, an amplification condition is: 60-66 degrees Celsius (° C.).

Optionally, Tm values of the external primers and the internal primers are 50-70° C.

Optionally, a length from a 5′ end of the LIF to a 5′ end of the LIR is 60-160 bp.

Optionally, a length from a 3′ end of the LOF to the 5′ end of the LIF is 0-60 bp.

Optionally, a length of a target sequence is 100-200 bp.

Optionally, a GC content of the target sequence is 35-70%.

Between 60° C. and 70° C., the double-stranded DNA is in the process of dissociation and semi-dissociation, so under the action of BST DNA polymerase, the double-stranded DNA may be combined with primers without being completely unbound (denatured at 95° C.), thus eliminating the need for denaturation and annealing of traditional PCR.

It is because there is no temperature change process that: 1: the reaction saves the time required for heating and cooling; 2: isothermal amplification is in a state of continuous reaction, and PCR starts extension only when the system is near 72° C., so the isothermal amplification reaction efficiency in the present invention is higher.

Optionally, a primer set sequence includes any one of (A) to (C):

(A)
external primers:
gE-LOF:
(SEQ ID NO. 6)
ACGAGCCCCGCTTCCA;
gE-LOR:
(SEQ ID NO. 7)
AGATGCAGGGCTCGTACA;
internal primers:
gE-LIF:
(SEQ ID NO. 1)
CGCGCTCGGCTTCCACT;
a sequence of gE-LIR is:
(SEQ ID NO. 2)
AGACCACGCGCGGCATCAG;
or
(SEQ ID NO. 3)
GCGCGAGTCGCCCATGTC;
or
(SEQ ID NO. 4)
AGCGTGGCGGTAAAGTTCT;
or
(SEQ ID NO. 5)
CGTAGTACAGCAGGCACCG;
accelerating primer:
LAR:
(SEQ ID NO. 8)
TGTCCCCGGGCGAGAAGA;
(B)
external primers:
LOF:
(SEQ ID NO. 17)
CTGGATGATGATTGGTTCAG;
LOR:
(SEQ ID NO. 18)
GAAGGGACGCTATGTCGA;
internal primer:
LIF:
(SEQ ID NO. 13)
TTATCAGATACCTATGCATACCCA;
a sequence of LIR is:
(SEQ ID NO. 14)
TGAACATGAGCTTTTCTTTATCGC;
or
(SEQ ID NO. 15)
AACATCATCTTCCCGATA;
or
(SEQ ID NO. 16)
TCCGGGTAATTTCTTCAACATC;
accelerating primer:
LAR:
(SEQ ID NO. 19)
TACAAATAATCGCCCGTAGCTGAT;
(C)
external primers:
LOF:
(SEQ ID NO. 24)
GGCCCTCGCATCCCTGA;
LOR:
(SEQ ID NO. 25)
ACGCGGTCTCGAAGCA;
internal primer:
LIF:
(SEQ ID NO. 22)
TGGTGAACGTGTCCGAGGGC;
a sequence of LIR is:
(SEQ ID NO. 23)
CGGGCAGGAACGTCCAGATC.

Optionally, the target sequence is a DNA or an RNA sequence.

Another aspect of the present disclosure provides a primer set, including the primers described in an aspect of the present disclosure.

Another aspect of the present disclosure provides a detection product, including the primer set described in an aspect of the present disclosure.

Optionally, the detection product includes a fluorescent probe/fluorescent dye.

Optionally, the fluorescent probe includes an OSD probe.

Optionally, the LAR primer is further extended and labeled with a fluorescent group at the 5′ end, and a complementary primer labeled with a quenching group at the 3′ end is designed to form an OSD probe.

Optionally, sequences of the OSD probe are:

gE-LAR-probe:
(SEQ ID NO. 9)
ATCAGGTCGAACGTGTCCCCGGGCGAGAAGA;
gE-LAR-quencher:
(SEQ ID NO. 10)
GGGGACACGTTCGACCTGAT.

Optionally, the fluorescent dye includes any one of Eva Green, SYBR Green and SYTO9.

Another aspect of the present disclosure provides an application of the primer set described in one aspect of the present disclosure or the product described in one aspect of the present disclosure in field detection.

Optionally, the application includes detection of drug-resistant genes of microorganisms, viruses and bacteria, species identification, gene screening and the like.

The present disclosure has the following beneficial effects.

The present disclosure provides a linear displacement isothermal amplification method, whereby an initial reaction of linear displacement isothermal amplification is initiated with four common primers for the template, including the external primers (LOF and LOR) and the internal primers (LIF and LIR), and the accelerating primer (LAR) may also be added to the reaction to form a short sequence product. In addition to substantially reducing the difficulty of primer design, the method of the present disclosure is capable of maintaining similar sensitivity and specificity as other isothermal amplification reactions such as loop-mediated isothermal amplification (LAMP) methods; it may be applied to target sequences with a higher GC content as well as those shorter than 200 bp. In the field detection, especially for complex nucleic acid sequences, the method of the present disclosure has a very good application prospect.

### BRIEF DESCRIPTION OF THE DRAWINGS

FIG. 1 shows the reaction principle of linear displacement isothermal amplification (LDIA).

FIG. 2 illustrates the effect of external primers on the reaction. Notes: M: 50 bp ladder marker; 1: reaction system containing external primers; 2. reaction system without external primers.

FIG. 3 shows the sequencing results of the reaction products.

FIG. 4 shows the reaction with different internal primers in the presence of external primers. Notes: 1: LIR1; 2: LIR2; 3: LIR3; 4: LIR4.

FIG. 5 shows the reaction with different internal primers without external primers. Notes: 1: LIR1; 2: LIR2; 3: LIR3; 4: LIR4.

FIG. 6 shows LAR acceleration effect. Notes: 1: accelerating primer is added; 2: no accelerating primer is added.

FIG. 7 shows the results of optimizing reaction temperature.

FIG. 8A shows the sensitivity test of LDIA reaction. Notes: 1-7: plasmid templates with copy numbers of 10⁶, 10⁵, 10⁴, 10³, 10², 10 and 1; 8: negative control.

FIG. 8B shows the LAMP sensitivity test. Notes: 1-7: plasmid templates with copy numbers of 10⁶, 10⁵, 10⁴, 10³, 10², 10 and 1; 8: negative control.

FIG. 9 shows the specificity test of LDIA reaction. Notes: 1: LAMP amplification curve with 10⁶ copies of plasmid template; 2. LAMP method negative control group; 3: LDIA method amplification curve of plasmid template with 10⁶ copies; 4: the negative control group of LDIA method.

FIG. 10 shows the reaction with different internal primers. Notes: 1: LIR1; 2: LIR2; 3: LIR3.

FIG. 11 shows the LAR acceleration effect. Notes: 1: accelerating primer is added; 2: no accelerating primer is added.

FIG. 12A shows the LDIA sensitivity test. Notes: 1-7: plasmid templates with copy numbers of 10⁶, 10¹, 10⁴, 10³, 10², 10 and 1.

FIG. 12B shows the LAMP sensitivity test. Notes: 1-7: plasmid templates with copy numbers of 10⁶, 10¹, 10⁴, 10³, 10², 10 and 1.

FIG. 13 shows the specificity test of LDIA reaction. Notes: 1: LAMP amplification curve of plasmid template with number of 10⁶; 2: negative control group of LAMP method; 3: LDIA amplification curve of plasmid template with number of 10⁶; 4. negative control group of LDIA method.

FIG. 14 shows the application of OSD probe in LDIA method.

FIG. 15 is a comparison between OSD probe primers and original NAR primers; Notes: 1: LAR; 2: OSD probe primer.

FIG. 16 shows the sensitivity test of LDIA by OSD probe method. Notes: 1-7: plasmid templates with numbers of 10⁶, 10¹, 10⁴, 10³, 10², 10 and 1; 8: negative control.

FIG. 17 shows illustrates a process of the linear displacement isothermal amplification (LDIA) method provided by the present disclosure.

### DETAILED DESCRIPTION OF THE EMBODIMENTS

In the following, the concept and technical effects of the present disclosure are described clearly and completely with embodiments, so as to fully understand the objectives, characteristics and effects of the present disclosure. Obviously, the described embodiments are only a part of the embodiments of the present disclosure, but not all the embodiments. Based on the embodiments of the present disclosure, other embodiments obtained by those skilled in the art without creative labor belong to the scope of protection of the present disclosure.

### Embodiment 1

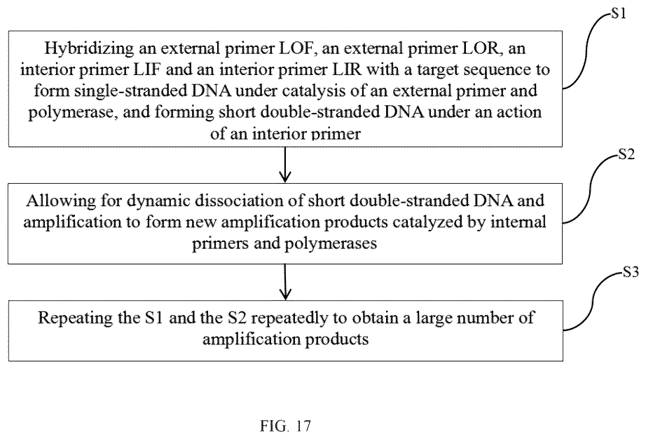

The present disclosure constructs a linear displacement isothermal amplification capable of detecting nucleic acid fragments with different lengths and different GC contents and simplifies the primer process, including the following steps as shown in FIG. 17:

S1, hybridizing an external primer LOF, an external primer LOR, an internal primer LIF and an internal primer LIR with a target sequence to form single-stranded DNA under catalysis of an external primer and polymerase, and forming short double-stranded DNA under an action of an internal primer;

S2, allowing for dynamic dissociation of short double-stranded DNA and amplification to form new amplification products catalyzed by internal primers and polymerases; and

S3, repeating the S1 and the S2 repeatedly to obtain a large number of amplification products.

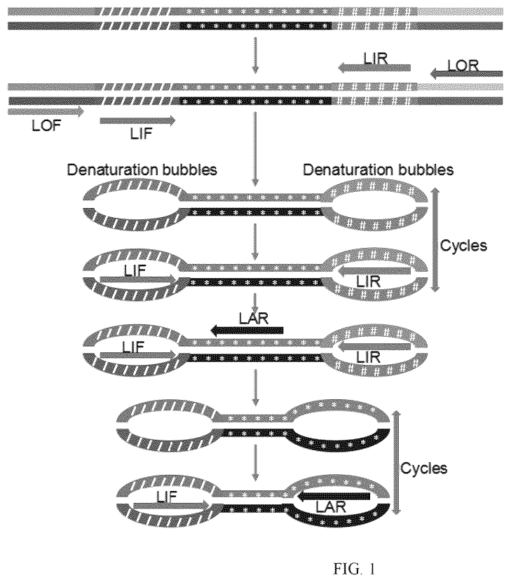

In this method, the initial reaction of LDIA is started with four common primers of the template, including a pair of external primers (LOF and LOR) and a pair of internal primers (LIF and LIR). The internal primers are easily bound to the template and amplified with the template since the concentration of the internal primers in the mixture is higher than that of the external primer. Single-stranded DNA (ssDNA) is formed with the help of an extended external primer and the strand displacement activity of BST DNA polymerase, and short double-stranded DNA (dsDNA) is formed by means of an internal primer. At a temperature of 60° C., these short strands of DNA (40-120 bp) undergo the process of double-stranded DNA respiration, i.e., in a dissociated and semi-dissociated state. Subsequently, LIF and LIR anneal to the dsDNA and generate new amplicons, respectively. These dsDNAs will continuously become new templates and initiate cyclic reactions. According to this principle, an accelerating primer (LAR) is added to the reaction to form a shorter product (40-60 bp) with LIF or LIR, and more amplicons will then be generated (FIG. 1). The innovation of the present disclosure lies in simplifying the primers for isothermal amplification. In the prior art, the primers for isothermal amplification are required to have a special structure, whereas the applicant accidentally found that the linear primers which do not require a special structure may also realize isothermal amplification under a certain combination.

Primer design principles of LDIA method

1. Tm value: The effective initiation temperature is generally 5-10° C. above the Tm value. If the Tm value of the primer is estimated according to the formula Tm=4(G+C)+2(A+T), the Tm of the effective primer is 55-70° C., and the Tm value is close to 60° C. for the best conditions.

2. Stability of primer end: Gibbs free energy at 3′ end of all primers is ΔG≤−4 kcal/mol.

3. GC content of the target: the too high or too low GC content of the target is not conducive to initiating the reaction. The GC content of LOF/LOR/LIF/LIR primers should not be too different, and the content is between 35% and 70%.

4. Secondary structure: it is important to note that the primer itself is not capable of forming a secondary structure, especially for internal primers, and it is especially important that the primer is designed not to form a secondary structure. This is because secondary structures not only affect the efficiency of the reaction but also lead to some non-specific amplification. To prevent the formation of primer dimers, the 3′ end of the primer is required to be non-complementary. If judged manually, the continuous complementary bases of primers themselves or between primers cannot be greater than 3 bp. The 3′ end of the primer should be avoided to have 3 bases of G or 3 bases of C arranged in series, and the last base of the 3′ end should be selected as T, C, G, not A. If the primer dimer and hairpin structure are unavoidable, the ΔG value should not be too low (should be higher than −4.5 kcal/mol).

5. Distance between primers: the length from the 5′ end of LIF to the 5′ end of LIR is 60-160 bp, and the length from the 3′ end of LOF to the 5′ end of LIF is 0-60 bp. The position of LAR is between LIF and LIR, and the area where LAR binds to the target does not coincide with the area where LIF and LIR bind to the target.

Primers of LDIA method may be designed by using Primer Premier 5, a common PCR design software. Under the condition that the Tm value of the primers is close to 60° C. and no primer dimer and secondary structure are generated, the target with a length of 100-200 bp and a GC content of 35%-70% may be amplified. In contrast, the primer of LAMP method needs to screen six regions of the target, and the length of the primer itself and the distance between primers make the primer design more complicated, so it is difficult to design a primer set for amplification of the target sequence shorter than 200 bp. In addition, LAMP method is also difficult to design primers when facing the target sequences with high GC content (more than 60%) and low GC content (less than 40%). Therefore, compared with LAMP, the primer design method in the present disclosure is simpler and may be used to detect shorter target sequences.

### Embodiment 2

1. The applicant designed the primer set of LDIA method with the gE gene (GenBank: KT936468.1) of pseudorabies virus (PRV) as the target gene (Table 1).

LDIA and LAMP systems: the concentration of each component in each 25 μL system is as follows: 1×Thermopol Isothermal buffer, 1×Eva Green, 1.6 mM dNTPs, 8 U Bst WarmStart DNA polymerase. The concentrations of LDIA primers are as follows: 1.6 μM LIF/LIR, 0.8 μM LAR and 0.2 μM LOF/LOR. The concentrations of LAMP primers are as follows: 1.6 μM FIP/BIP, 0.8 μM LF and 0.2 μM F3/B3. Both LDIA reaction and LAMP reaction are carried out at 63° C. for 60 minutes.

TABLE 1
Primer groups of LDIA method and LAMP method for PRV gE gene

| Primer name | Sequences 5′-3′ | Genome position |
|---|---|---|
| gE-LIF | CGCGCTCGGCTTCCACT (SEQ ID NO. 1) | 684-700 |
| gE-LIR1 | AGACCACGCGCGGCATCAG (SEQ ID NO. 2) | 733-751 |
| gE-LIR2 | GCGCGAGTCGCCCATGTC (SEQ ID NO. 3) | 754-771 |
| gE-LIR3 | AGCGTGGCGGTAAAGTTCT (SEQ ID NO. 4) | 773-791 |
| gE-LIR4 | CGTAGTACAGCAGGCACCG (SEQ ID NO. 5) | 820-838 |
| gE-LOF | ACGAGCCCCGCTTCCA (SEQ ID NO. 6) | 668-683 |
| gE-LOR | AGATGCAGGGCTCGTACA (SEQ ID NO. 7) | 839-856 |
| gE-LAR | TGTCCCCGGGCGAGAAGA (SEQ ID NO. 8) | 707-724 |
| gE-LAR- | ATCAGGTCGAACGTGTCCCCGGGCGAGAAGA | 707-737 |
| probe | (SEQ ID NO. 9) | |
| gE-LAR- | GGGGACACGTTCGACCTGAT (SEQ ID NO. 10) | 718-737 |
| quencher | | |
| gE-FIP | AGACCACGCGCGGCATCAG-GCGCTCGGCTTC | F1c: 733-751 |
| (F1c + F2) | CACT (SEQ ID NO. 11) | F2: 685-700 |
| gE-BIP | GAGAACTTTACCGCCACGCTGG-CGTAGTACA | B1c: 772-793 |
| (B1c + B2) | GCAGGCACCG (SEQ ID NO. 12) | B2: 820-838 |
| gE-LF | TGTCCCCGGGCGAGAAGA (SEQ ID NO. 8) | 707-724 |
| gE-F3 | ACGAGCCCCGCTTCCA (SEQ ID NO. 6) | 668-683 |
| gE-B3 | AGATGCAGGGCTCGTACA (SEQ ID NO. 7) | 839-856 |

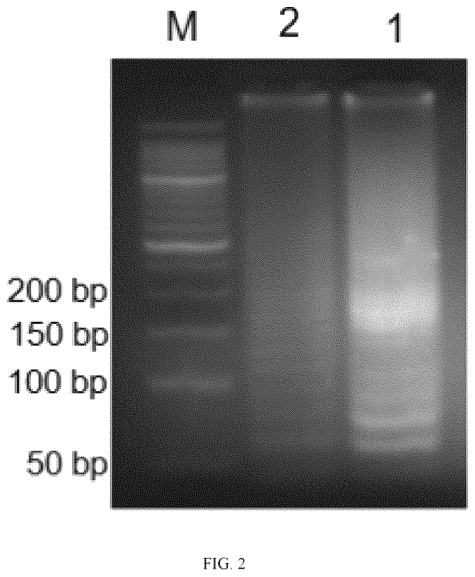

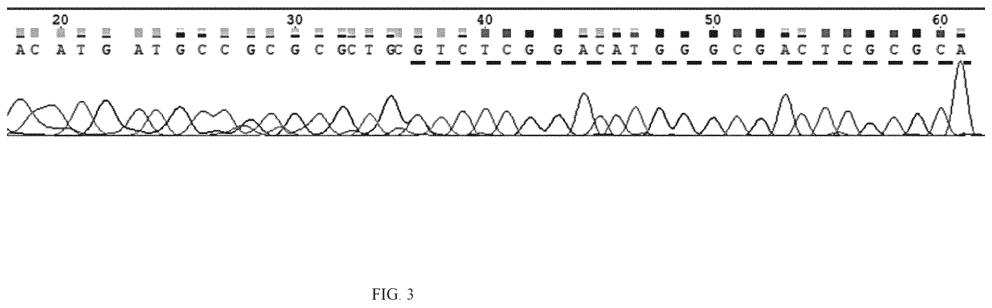

Firstly, the necessity of external primers for LDIA method is evaluated. The applicant uses Eva Green dye to monitor the reaction in real time. In the presence of external primers, the reaction proceeds normally, but in the absence of external primers, the reaction fails to produce amplification signals. The reaction products are analyzed by PAGE gel electrophoresis, and a band with expected size (80 bp) and subsequent stepped bands are found (FIG. 2). Subsequently, the reaction principle is further verified by sequencing the 80 bp reaction product, and the sequence is in line with the expectation (FIG. 3). This shows that the external primer is essential for the normal reaction.

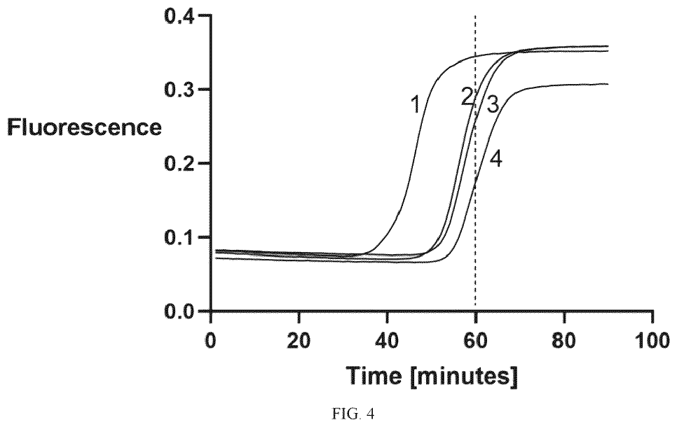

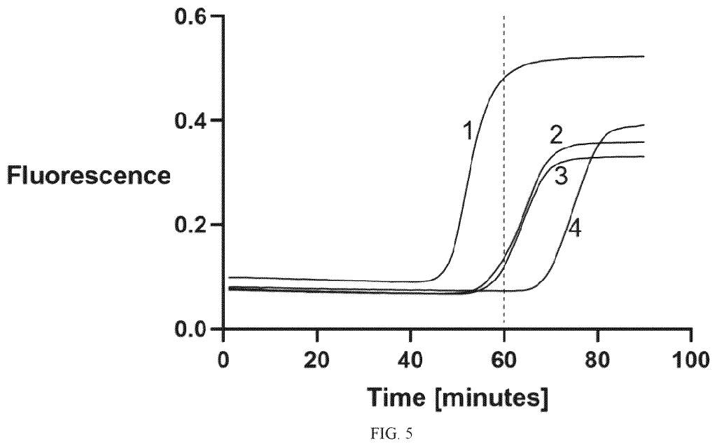

Secondly, the applicant analyzes the applicability of LDIA to target genes with different lengths. The length of products produced by LIF and LIR1, LIR2, LIR3 and LIR4 gradually increases, and the product length is 67 (751 minus 684, that is, from the 5′ end of LIF to the 5′ end of LIR1), 87, 107 and 155 bp respectively. It is observed that when the length of the reaction product is 155 bp, the reaction proceeds smoothly still (FIG. 4). However, when the reaction has no external primer involved, the reaction is difficult to start once the reaction product exceeds 100 bp (FIG. 5).

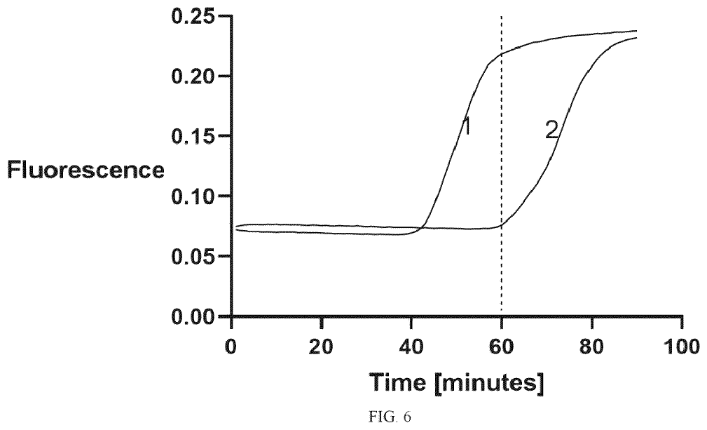

In order to improve the reaction efficiency of LDIA, the applicant tries to promote it by additional accelerating primers. According to the principle of LDIA, it is speculated that the reaction may be accelerated by the enhancement of short product formation or the formation of denatured bubbles in dsDNA. By adding short-chain products formed by accelerating primers LAR and LIF, denatured bubbles are easily formed, and the cycle threshold (CT) of LDIA reaction signal reaching the threshold is significantly reduced (FIG. 6). This proves that further addition of accelerating primer is effective in accelerating LDIA, which is consistent with the applicant's conjecture that shorter products are favorable for improving the efficiency of the LDIA reaction.

2. Effect of reaction temperature on LDIA method

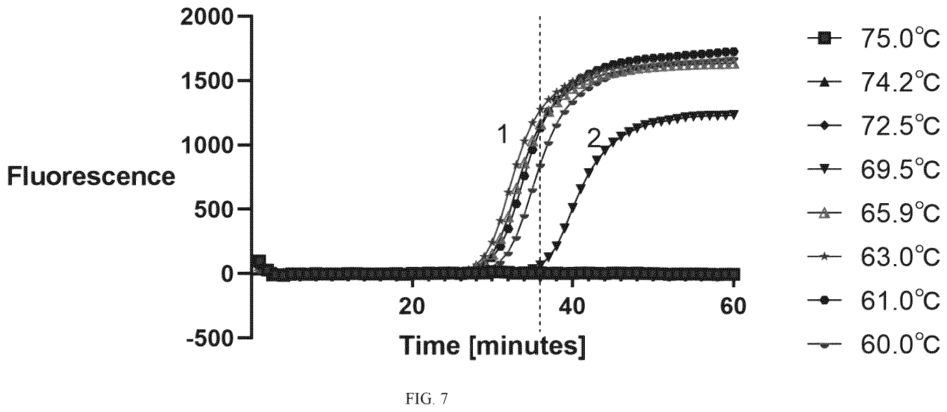

The applicant analyzes the influence of high temperature on LDIA, and it is reported that LDIA plays an active role in the formation of denatured bubbles in dsDNA. Considering that the inactivation temperature of Bst DNA polymerase is about 80° C., in order to balance high temperature and enzyme activity, the applicant sets the test temperature range as 60-75° C. The reaction results show that 63° C. is the optimum temperature for the reaction (FIG. 7).

### Embodiment 3 Sensitivity and Specificity Tests of LDIA Method

### (1) Sensitivity

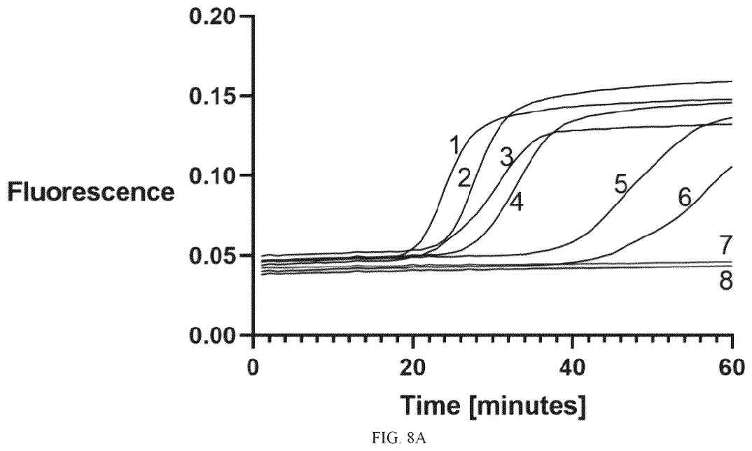

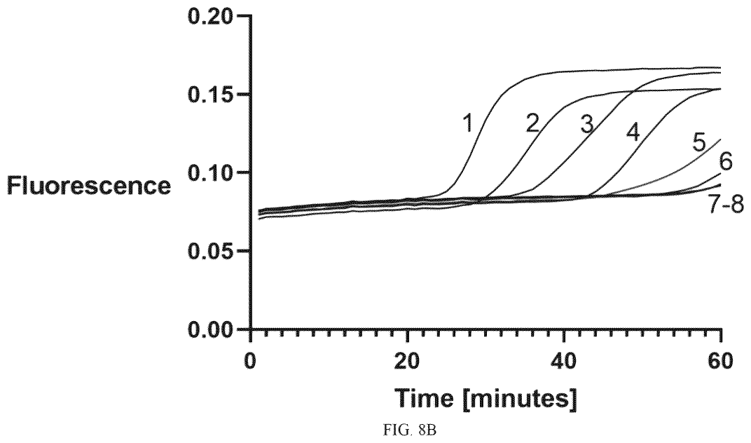

The applicant uses 10-fold gradient diluted dsDNA as a template to evaluate the sensitivity of LDIA. The primer information is shown in Table 1, and the internal primer used is LIR4. At the same time, the LAMP method for the same region of the gene is designed for comparison. Since the LAMP method is not suitable for amplifying target genes that are too short, the target gene length of this LAMP method (Table 1) is 200 bp, and the results indicate that the lowest detection limit of LDIA is 100 copies/μL, which is comparable to the sensitivity of LAMP (FIG. 8A and FIG. 8B).

### (2) Specificity

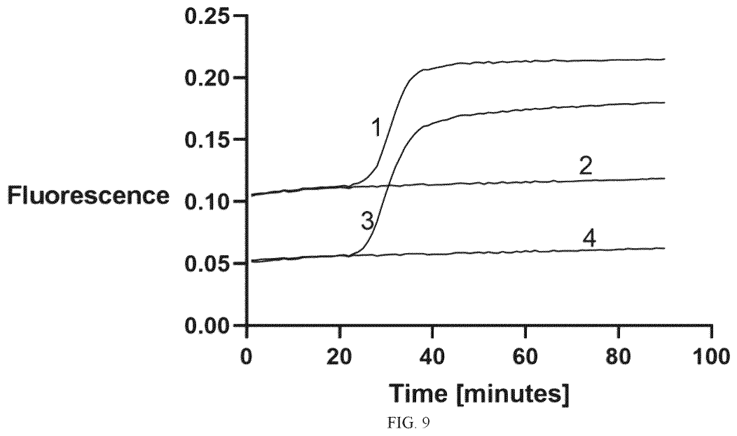

The specificity of LDIA is tested by the applicant and is compared with the LAMP method, and the results show that the negative group does not produce non-specific signals under prolonged incubation and exhibits good specificity in the case of a normal amplification reaction in the positive group (FIG. 9). Therefore, the sensitivity and specificity of LDIA method are comparable to those of LAMP method. In addition, the negative group of the LDIA method exhibits a lower background signal than the negative group of the LAMP method, which may be related to its simple primer composition, since LAMP requires loop-forming primers (FIP\BIP), and those in LDIA are all linear primers, and the design conditions are easier to be met than loop-forming primers.

### (3) Primer Design

The applicant finds it more difficult to design LAMP primers targeting the gE gene using the online PrimerExplorer software. As the gene has a high GC content, only 2 sets of suitable PRV LAMP primers may be generated using the software. Therefore, another advantage of LDIA over LAMP is the simplicity of the primer design process.

### (4) Universal Testing

Subsequently, the fimW gene of Salmonella is detected by LDIA method (GenBank: 1252072) to verify the universality of LDIA method. Primers of LDIA method are designed for fimW gene (Table 2).

TABLE 2
Primer groups for fimW gene of Salmonella by LDIA method and LAMP method

| Primer | | Gene |
|---|---|---|
| name | Sequences 5′-3′ | position |
| fim W-LIF | TTATCAGATACCTATGCATACCCA (SEQ ID NO. | 183-206 |
| | 13) | |
| fim W-LIR1 | TGAACATGAGCTTTTCTTTATCGC (SEQ ID NO. | 239-262 |
| | 14) | |
| fim W-LIR2 | AACATCATCTTCCCGATA (SEQ ID NO. 15) | 292-309 |
| fim W-LIR3 | TCCGGGTAATTTCTTCAACATC (SEQ ID NO. 16) | 304-325 |
| fim W-LOF | CTGGATGATGATTGGTTCAG (SEQ ID NO. 17) | 154-173 |
| fimW-LOR | GAAGGGACGCTATGTCGA (SEQ ID NO. 18) | 357-374 |
| fimW-LAR | TACAAATAATCGCCCGTAGCTGAT (SEQ ID NO. | 209-232 |
| | 19) | |
| fim W-FIP | ACATGAGCTTTTCTTTATCGCATT-TTATCAGA | F2: 183-206, |
| (F1c + F2) | TACCTATGCATACCCA (SEQ ID NO. 20) | Flc: 236-259 |
| fim W-BIP | CAGACCATGTCTGTATATGCTGCC-TGTAAGAT | B2: 321-344 |
| (B1c + B2) | CAATATCATTTTCCGG (SEQ ID NO. 21) | B1c: 261-284 |
| fim W-LF | TACAAATAATCGCCCGTAGCTGAT (SEQ ID NO. | 209-232 |
| | 19) | |
| fimW-F3 | CTGGATGATGATTGGTTCAG (SEQ ID NO. 17) | 154-173 |
| fimW-B3 | GAAGGGACGCTATGTCGA (SEQ ID NO. 18) | 357-374 |

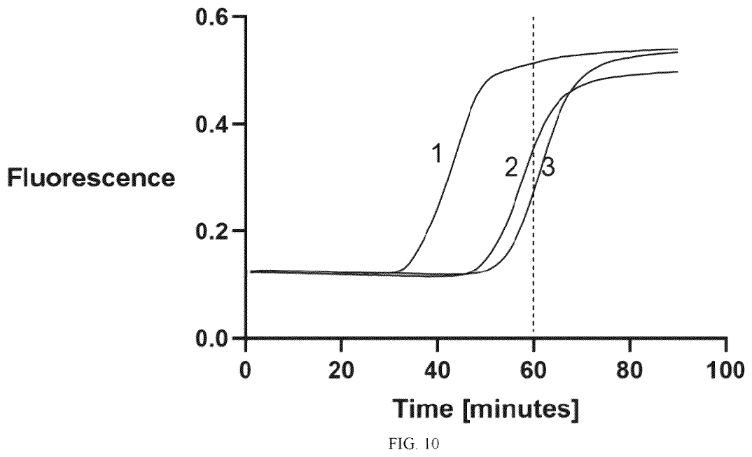

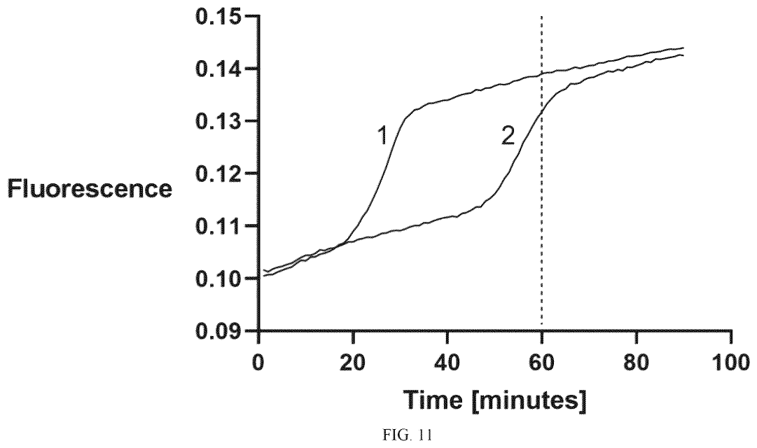

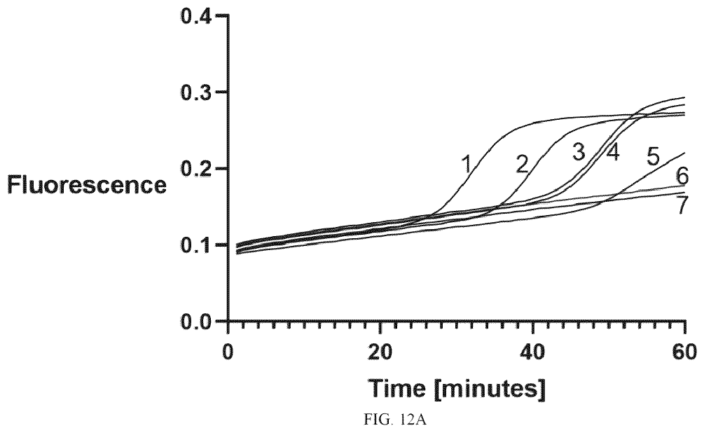

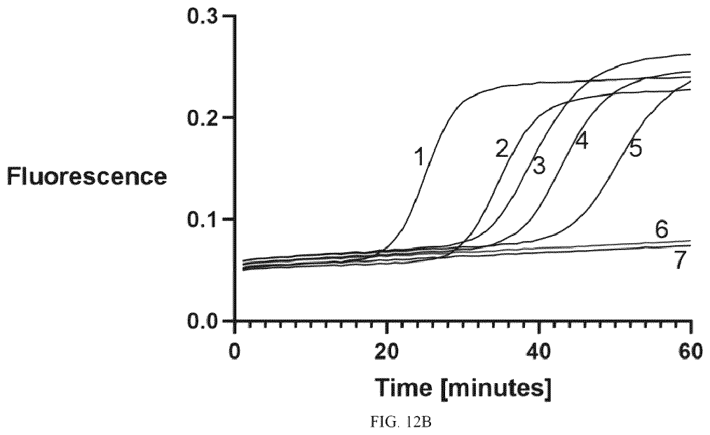

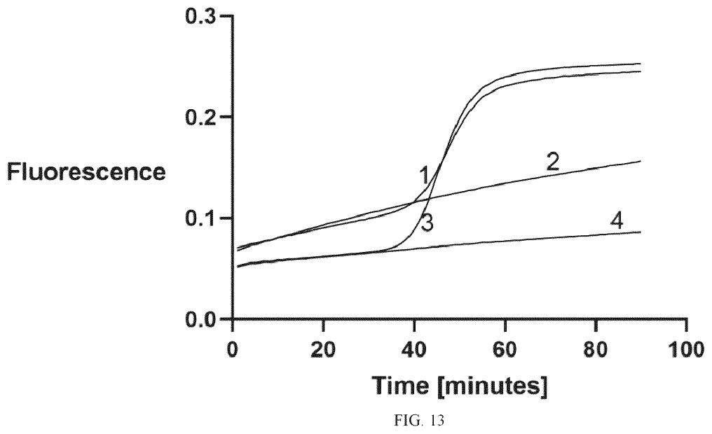

It is demonstrated that the LDIA method amplifies sequences of 80 bp-140 bp length (FIG. 10). Similarly, the accelerating primer LAR gives a significant boost to the reaction (FIG. 11). The LDIA method achieves the same sensitivity as the LAMP method (FIG. 12A and FIG. 12B) with good specificity (FIG. 13).

### Embodiment 3 LDIA Method Combining Fluorescent Probes

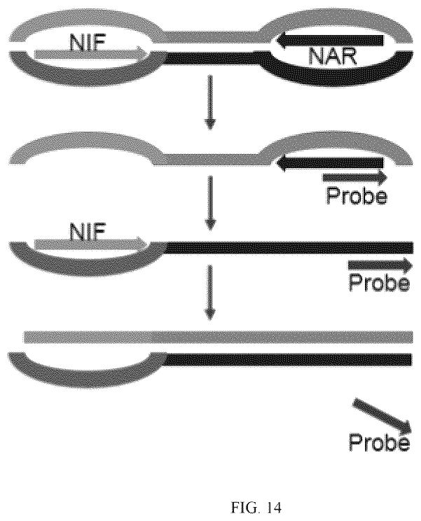

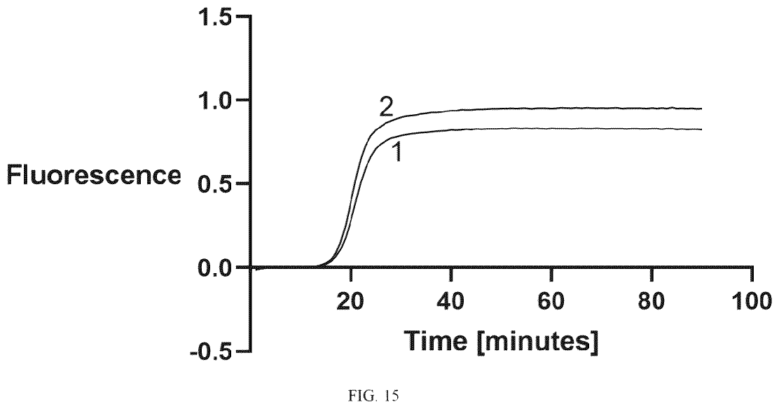

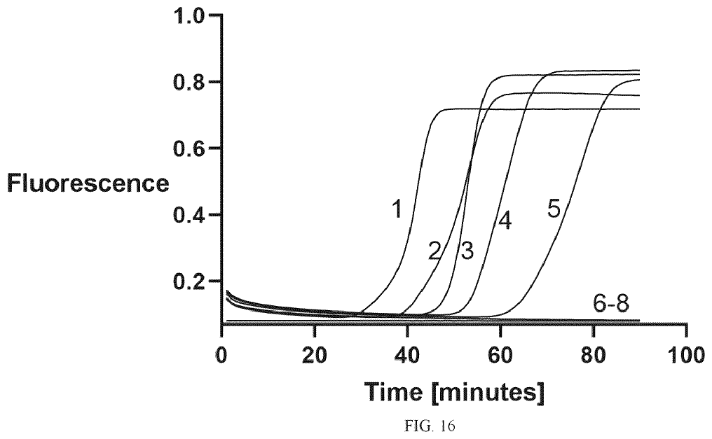

Considering that all primers in the LDIA method have a common linear structure, the applicant thinks that oligonucleotide strand exchange (OSD) probes may be well combined with them. As shown in FIG. 15, the reaction is not inhibited by increasing the length of the LAR primer to 30 bp. The 31 bp LAR primer is named as gE-LAR-probe (ATCAGGTCGAACGTGTCCCCGGGCGAGAAGA (SEQ ID NO. 9)), and the 5′ end is labeled with FAM fluorescent group. A 20 bp complementary primer (gE-LAR-quencher) (GGGGACACGTTCGACCTGAT (SEQ ID NO. 10)) is labeled with a BHQ1 quenching motif at the 3′ end to form a pair of OSD probes (Table 1). Due to the strong complementarity between these two probes, only a stable amplification allows for the exchange of quenching probes and the generation of a signal output (FIG. 14). A typical amplification profile is detected in the LDIA method when the OSD probe is added to the reaction system. The sensitivity is the same as that of the Eva Green dye method (FIG. 16).

### Embodiment 4 Design of LDIA Primer for 200 bp Target Sequence of gE Gene

### (1) Design of LDIA Primers for Short Sequence Targets

A 200 bp-sized sequence in the gE gene (with sequence of SEQ ID NO. 29 as shown in Table 4) is subjected to LAMP primer design and LDIA primer design, respectively. The LAMP primer design software Primer Explorer v5 fails to design suitable primers under default parameters, while 10 pairs of upstream and downstream primers are available using the PCR primer design software Primer Premier 5 under default parameters, in which a set of LDIA primers may be easily screened according to the LDIA primer design principles (as shown in Table 3).

TABLE 3
Primer set of LDIA method targeting gE gene

| Primer | |
|---|---|
| name | Primer sequences (5′-3′) |
| LIF | TGGTGAACGTGTCCGAGGGC (SEQ ID NO. 22) |
| LIR | CGGGCAGGAACGTCCAGATC (SEQ ID NO. 23) |
| LOF | GGCCCTCGCATCCCTGA (SEQ ID NO. 24) |
| LOR | ACGCGGTCTCGAAGCA (SEQ ID NO. 25) |

TABLE 4
Sequences used for primer design

| Sequence name | Primer sequences (5′-3′) |
|---|---|
| A 200 bp | GCCGCGCGGGCTTCGGCTCGGCCCTCGCATCCCTGAGGGAGGC |
| sequence in gE | GCCCCCGGCCCATCTGGTGAACGTGTCCGAGGGCGCCAACTTCA |
| gene of PRV | CCCTCGACGCGCGCGGCGACGGCGCCGTGCTGGCCGGGATCTG |
| | GACGTTCCTGCCCGTCCGCGGCTGCGACGCCGTGTCGGTGACCA |
| | CGGTGTGCTTCGAGACCGCGTGCCAC (SEQ ID NO. 29) |
| gE gene of PRV | ATGCGGCCCTTTCTGCTGCGCGCCGCGCAGCTCCTGGCGCTGCT |
| | GGCCCTGGCGCTCTCCACCGAGGCCCCGAGCCTCTCCGCCGAGA |
| | CGACCCCGGGCCCCGTCACCGAGGTCCCGAGTCCCTCGGCCGA |
| | GGTCTGGGACGACCTCTCCACCGAGGCCGACGACGATGACCTC |
| | AACGGCGACCTCGACGGCGACGACCGCCGCGCGGGCTTCGGCT |
| | CGGCCCTCGCATCCCTGAGGGAGGCGCCCCCGGCCCATCTGGTG |
| | AACGTGTCCGAGGGCGCCAACTTCACCCTCGACGCGCGCGGCG |
| | ACGGCGCCGTGCTGGCCGGGATCTGGACGTTCCTGCCCGTCCGC |
| | GGCTGCGACGCCGTGTCGGTGACCACGGTGTGCTTCGAGACCG |
| | CGTGCCACCCGGACCTGGTGCTGGGCCGCGCCTGCGTCCCCGAG |
| | GCCCCGGAGATGGGCATCGGCGACTACCTGCCGCCCGAGGTGC |
| | CGCGGCTCCGGCGCGAGCCGCCCATCGTCACCCCGGAGCGGTG |
| | GTCGCCGCACCTGAGCGTCCTGCGGGCCACGCCCAACGACACG |
| | GGCCTCTACACGCTGCACGACGCCTCGGGGCCGCGGGCCGTGTT |
| | CTTTGTGGCGGTGGGCGACCGGCCGCCCGCGCCGGCGGACCCG |
| | GTGGGCCCCGCGCGCCACGAGCCCCGCTTCCACGCGCTCGGCTT |
| | CCACTCGCAGCTCTTCTCGCCCGGGGACACGTTCGACCTGATGC |
| | CGCGCGTGGTCTCGGACATGGGCGACTCGCGCGAGAACTTTAC |
| | CGCCACGCTGGACTGGTACTACGCGCGCGCGCCCCCGCGGTGC |
| | CTGCTGTACTACGTGTACGAGCCCTGCATCTACCACCCGCGCGC |
| | GCCCGAGTGCCTGCGCCCGGTGGACCCGGCGTGCAGCTTCACCT |
| | CGCCGGCGCGCGCGCGGCTGGTGGCGCGCCGCGCGTACGCCTC |
| | GTGCAGCCCGCTGCTCGGGGACCGGTGGCTGACCGCCTGCCCCT |
| | TCGACGCCTTCGGCGAGGAGGTGCACACGAACGCCACCGCGGA |
| | CGAGTCGGGGCTGTACGTGCTCGTGATGACCCACAACGGCCAC |
| | GTCGCCACCTGGGACTACACGCTCGTCGCCACCGCGGCCGAGT |
| | ACGTCACGGTCATCAAGGAGCTGACGGCCCCGGCCCGGGCCCC |
| | GGGCACCCCGTGGGGCCCCGGCGGCGGCGACGACGCGATCTAC |
| | GTGGACGGCGTCACGACGCCGGCGCCGCCCGCGCGCCCGTGGA |
| | ACCCGTACGGCCGGACGACGCCCGGGCGGCTGTTTGTGCTGGC |
| | GCTGGGCTCCTTCGTGATGACGTGCGTCGTCGGGGGGGCCATCT |
| | GGCTCTGCGTGCTGTGCTCCCGGCGCCGGGCGGCCTCGCGGCCG |
| | TTCCGGGTGCCGACGCGGGCGCGGACGCACATGCTCTCTCCGGT |
| | GTACACCAGCCTGCCCACGCACGAGGACTACTACGACGGCGAC |
| | GACGACGACGACGAGGAGGCGGGCGTCATCCGCCGGCGGCCCG |
| | CCTCCCCCGGCGGAGACAGCGGCTACGAGGGGCCGTACGCGAG |
| | CCTGGACCCCGAGGACGAGTTCAGCAGCGACGAGGACGACGGG |
| | CTGTACGTGCGCCCCGAGGAGGCGCCCCGCTCCGGCTTCGACGT |
| | CTGGTTCCGCGATCCGGAGAAACCGGAAGTGACGAATGGACCC |
| | AACTATGGCGTGACCGCCAACCGCCTGTTGATGTCCCGCCCCGC |
| | TTAA (SEQ ID NO. 30) |
| A 250 bp sequence | ATCGCTTTCCTGGCCCTGGATGATGATTGGTTCAGCGCTGGCTG |
| in fimW gene of | TTATCAGATACCTATGCATACCCAACATCAGCTACGGGCGATTA |
| Salmonella | TTTGTAATAAATGCGATAAAGAAAAGCTCATGTTCAGACCATGT |
| | CTGTATATGCTGCCGCATATTTATCGGGAAGATGATGTTGAAGA |
| | AATTACCCGGAAAATGATATTGATCTTACATAAACGAGCGCTTC |
| | GACATAGCGTCCCTTCTGGCATTTGCCACT (SEQ ID NO. 31) |

(2) Design of LDIA Primers for Targets with High GC Content and Low GC Content (Aimed at GC Content)

The sequence of pseudorabies virus gE gene (GC content 74%, Table 4) is used to design LAMP primer and LDIA primer respectively. Only two sets of available primers may be designed using LAMP online software Primer Explorer v5 in the default mode, while 100 pairs of primer sets are available by using PCR primer design software Primer Premier 5 in the automatic search mode. According to the principle of LDIA primer design, 8 sets of suitable LDIA primer sets (LIF and LIR permutation and combination) are preliminarily selected (as shown in Table 4). A 250 bp (139-388) sequence of Salmonella fimW gene (GC content 42%, Table 4) is used for LAMP primer design and LDIA primer design. Only 3 sets of LAMP primers are designed by LAMP online software Primer Explorer v5 in the default mode, but 51 pairs of PCR primer sets may be designed by PCR primer design software Primer Premier 5 in the automatic search mode, and 9 sets of LDIA primer sets may also be obtained by simple screening (see Table 5-Table 6).

TABLE 5
Primer set of LDIA method for pseudorabies virus gE gene

| Primer name | Sequences 5′-3′ | Genome position |
|---|---|---|
| gE-LIF1 | CGCGCTCGGCTTCCACT (SEQ ID NO. 1) | 684-700 |
| gE-LIF2 | TCCACTCGCAGCTCTTCT (SEQ ID NO. 26) | 695-712 |
| gE-LIR1 | AGACCACGCGCGGCATCAG (SEQ ID NO. 2) | 733-751 |
| gE-LIR2 | GCGCGAGTCGCCCATGTC (SEQ ID NO. 3) | 754-771 |
| gE-LIR3 | AGCGTGGCGGTAAAGTTCT (SEQ ID NO. 4) | 773-791 |
| gE-LIR4 | CGTAGTACAGCAGGCACCG (SEQ ID NO. 5) | 820-838 |

TABLE 6
Primer set of LDIA method for Salmonella fimW gene

| Primer name | Sequences 5′-3′ | Gene position |
|---|---|---|
| fimW-LIF1 | TTATCAGATACCTATGCATACCCA (SEQ ID NO. 13) | 183-206 |
| fimW-LIF2 | CGCTGGCTGTTATCAGAT (SEQ ID NO. 27) | 174-191 |
| fimW-LIF3 | CTATGCATACCCAACATCAG (SEQ ID NO. 28) | 194-213 |
| fimW-LIR1 | TGAACATGAGCTTTTCTTTATCGC (SEQ ID NO. 14) | 239-262 |
| fimW-LIR2 | AACATCATCTTCCCGATA (SEQ ID NO. 15) | 292-309 |
| fimW-LIR3 | TCCGGGTAATTTCTTCAACATC (SEQ ID NO. 16) | 304-325 |

To sum up, compared with LAMP, the design method of primers in LDIA in the invention is simpler and more specific, and may be applied to target sequences with higher GC content and below 200 bp.

The above specific embodiments have explained the present disclosure in detail, but the present disclosure is not limited to the above embodiments, and various changes may be made within the knowledge of ordinary technicians in the technical field without departing from the purpose of the present disclosure. In addition, embodiments of the present disclosure and features in embodiments can be combined with each other without conflict.

What is claimed is:

1. A linear displacement isothermal amplification method, comprising following steps:
S1, hybridizing an external primer LOF, an external primer LOR, an internal primer LIF and an internal primer LIR with a target sequence to form single-stranded DNA under catalysis of an external primer and polymerase, and forming short double-stranded DNA under an action of an internal primer;
S2, allowing for dynamic dissociation of the short double-stranded DNA and amplification to form new amplification products catalyzed by internal primers and polymerases; and
S3, repeating the S1 and the S2 repeatedly to obtain a large number of amplification products.

2. The linear displacement isothermal amplification method according to claim 1, wherein an accelerating primer LAR is added in the S1; the LAR is positioned between the LIF and the LIR, and a region of LAR binding to a target does not overlap with a region of LIF and LIR binding to the target.

3. The linear displacement isothermal amplification method according to claim 1, wherein a molar ratio of the external primer LOF, the external primer LOR, the internal primer LIF, the internal primer LIR and the accelerating primer LAR is (1-2):(1-2):(4-10):(4-10):(3-4); and an amplification temperature is 60-66 degrees Celsius.

4. The linear displacement isothermal amplification method according to claim 1, wherein a length from a 5′ end of the LIF to a 5′ end of the LIR is 60-160 bp; and a length from a 3′ end of the LOF to the 5′ end of the LIF is 0-60 bp.

5. The linear displacement isothermal amplification method according to claim 1, wherein a hlength of a target sequence is 100-200 bp, and a GC content of the target sequence is 35-70%.

6. The linear displacement isothermal amplification method according to claim 1, wherein a primer set sequence includes any one of (A) to (C):
(A)
external primers:
gE-LOF:
(SEQ ID NO. 6)
ACGAGCCCCGCTTCCA;
gE-LOR:
(SEQ ID NO. 7)
AGATGCAGGGCTCGTACA;
internal primers:
gE-LIF:
(SEQ ID NO. 1)
CGCGCTCGGCTTCCACT;
a sequence of gE-LIR is:
(SEQ ID NO. 2)
AGACCACGCGCGGCATCAG;
or
(SEQ ID NO. 3)
GCGCGAGTCGCCCATGTC;
or
(SEQ ID NO. 4)
AGCGTGGCGGTAAAGTTCT;
or
(SEQ ID NO. 5)
CGTAGTACAGCAGGCACCG;
accelerating primer:
LAR:
(SEQ ID NO. 8)
TGTCCCCGGGCGAGAAGA;
(B)
external primers:
LOF:
(SEQ ID NO. 17)
CTGGATGATGATTGGTTCAG;
LOR:
(SEQ ID NO. 18)
GAAGGGACGCTATGTCGA;
internal primer:
LIF:
(SEQ ID NO. 13)
TTATCAGATACCTATGCATACCCA;
a sequence of LIR is:
(SEQ ID NO. 14)
TGAACATGAGCTTTTCTTTATCGC;
or
(SEQ ID NO. 15)
AACATCATCTTCCCGATA;
or
(SEQ ID NO. 16)
TCCGGGTAATTTCTTCAACATC;
accelerating primer:
LAR:
(SEQ ID NO. 19)
TACAAATAATCGCCCGTAGCTGAT;
(C)
external primers:
LOF:
(SEQ ID NO. 24)
GGCCCTCGCATCCCTGA;
LOR:
(SEQ ID NO. 25)
ACGCGGTCTCGAAGCA;
internal primer:
LIF:
(SEQ ID NO. 22)
TGGTGAACGTGTCCGAGGGC;
a sequence of LIR is:
(SEQ ID NO. 23)
CGGGCAGGAACGTCCAGATC.

7. A primer set, comprising the primers according to claim 1.

8. A detection product, comprising the primer set according to claim 7, wherein the detection product further comprises a fluorescent probe/fluorescent dye.

9. The detection product according to claim 8, wherein the fluorescent probe comprises an OSD probe; a preparation method of the OSD probe comprises: the LAR primer is further extended and labeled with a fluorescent group at the 5′ end, and a complementary primer labeled with a quenching group at the 3′ end is designed to form the OSD probe;
sequences of the OSD probe are:
gE-LAR-probe:
(SEQ ID NO. 9)
ATCAGGTCGAACGTGTCCCCGGGCGAGAAGA;
gE-LAR-quencher:
(SEQ ID NO. 10)
GGGGACACGTTCGACCTGAT.
